(19) United States
(12) Patent Application Publication
(10) Pub. No.: US 2016/0331428 A1
(43) Pub. Date: Nov. 17, 2016

(54) FLEXIBLE BONE DILATOR

(71) Applicant: SMITH & NEPHEW, INC., Memphis, TN (US)

(72) Inventor: Konsei Shino, Osaka (JP)

(73) Assignee: Smith & Nephew, Inc., Memphis, TN (US)

(21) Appl. No.: 15/112,546

(22) PCT Filed: Feb. 10, 2015
(86) PCT No.: PCT/US2015/015147

§ 371 Date: Jul. 19, 2016

Related U.S. Application Data
(60) Provisional application No. 61/938,215, filed on Feb. 11, 2014.

(51) Int. Cl. A61B 17/88

(52) U.S. Cl. CPC A61B 17/885; A61B 2017/00473

(57) ABSTRACT

A bone tunnel dilator (5) includes an elongate, flexible member (10) having leading (10L) and trailing (10T) portions, and at least one dilating element (12) disposed on a member intermediate the leading and trailing ends, a leading end of the shaft designed to be inserted through a bone tunnel and the at least one dilating element pulled into an entry end of the bone tunnel by applying pulling force to the leading end of the shaft to dilate an entry end of the bone tunnel to a shape and size defined by the dilating element, the dilator removed by pulling on the trailing end of the

### BACKGROUND

The present invention relates generally to bone tunnels, and more specifically to a flexible bone dilator.

In general, bone tunnel dilators are used to prepare a tunnel drilled within a bone in which the tunnel is adapted to receive an anchor for a graft, such as in repair of an anterior cruciate ligament (ACL) or other surgical procedure involving securing an anchor in a bone. When using a dilator, after the bone tunnel is drilled, the dilator is inserted into an end of the tunnel and is driven into the tunnel to dilate an end of the tunnel to a shape configured to receive the anchor selected for the particular procedure. The dilating element typically is formed on the leading end of a rigid shaft and is driven, in increments, into the tunnel end by repeatedly impacting the trailing end of the shaft, as with a hammer In addition to dilating and reshaping the drilled hole, the dilating process also compacts the cancellous bone to provide a more dense structure into which the anchor may become integrated.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention is directed towards a method and an apparatus for tissue graft fixation with tension adjustment.

In an aspect, the invention features bone tunnel dilator including an elongate, flexible member having leading and trailing portions, and at least one dilating element disposed on a member intermediate the leading and trailing ends, a leading end of the shaft designed to be inserted through a bone tunnel and the at least one dilating element pulled into an entry end of the bone tunnel by applying pulling force to the leading end of the shaft to dilate an entry end of the bone tunnel to a shape and size defined by the dilating element, the dilator removed by pulling on the trailing end of the shaft.

In another aspect, the invention features a method including providing a bone dilator having an elongate, flexible member having leading and trailing portions and a dilating element disposed on the member, intermediate ends of the flexible member, passing the leading portion of the member through a bone tunnel until the dilating element is in engagement with an entry end of the bone tunnel and the leading portion of the member extends out of a patient, pulling the bone dilator by the leading portion of the member, and in increments, to draw the dilating element into the bone tunnel to dilate the bone tunnel to a cross-sectional shape of the dilating element, and removing the dilator out of the dilated tunnel by pulling the trailing portion of the flexible member in a removal direction.

Embodiments of the invention may have one or more of the following advantages.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The invention will be more fully understood by reference to the detailed description, in conjunction with the following figures, wherein:

FIG. 1 is an illustration of an exemplary bone dilator.

FIG. 2 is an illustration the ridges of the bone dilator of FIG. 1.

FIG. 3 is a flow diagram of dilating a bone tunnel.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

In the description below, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

A dilator serves to enlarge and change a cross sectional shape of a bone tunnel drilled into a bone from a circular cross section left by the bone drill to a rectangular or other desired cross section that is better adapted to receive a rectangular or other shape bone anchoring plug to which one end of a prosthesis is attached. The prosthesis may be a replacement anterior cruciate ligament (ACL), although the device of the present invention may be usable in other orthopaedic applications.

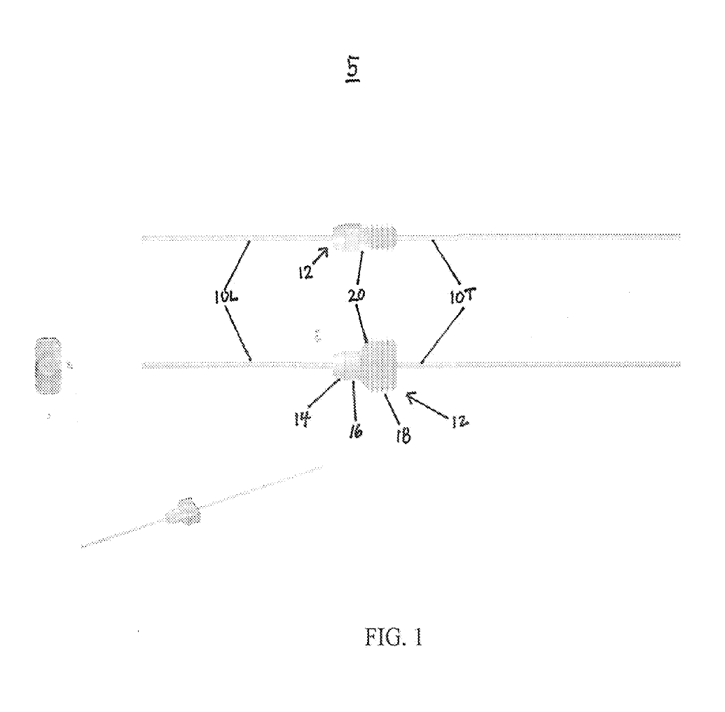

FIG. 1 illustrates an exemplary bone dilator 5 in side, top, end and isometric views. The dilator 5 includes an elongate, flexible shaft 10 having leading and trailing segments, 10L and 10T, respectively, and a dilator element, indicated generally at 12, located between the leading and trailing shaft segments 10L, 10T. The flexible shaft 10 and dilator element 12 may be formed as an integral, single unit and may be made from a single mass of material such as surgical stainless steel or other suitable metal or a polymer of sufficient hardness to perform dilating and smoothing functions. Alternately, the dilator 5 may be made from components attached to each other to form the complete device.

The dilator element 12 includes a leading frusto-conical section 14 having a diameter equal to or slightly larger than that of a drilled bone tunnel, a cylindrical midsection 16 with a diameter equal to the largest diameter of the frusto-conical section and a trailing portion 18. The trailing portion 18, in the illustrative embodiment, is approximately rectangular in cross section but may have radiused corners so that a cross section may be considered as slightly oval. A transition region 20 is formed at the leading end of the trailing portion 18 of the dilator element 12 to progressively expand the cross-sectional dimensions of the dilator element 12 (in a trailing direction) from a circular shape to an intended larger rectangular (oval) shape. The trailing portion 18 of the dilator element 12 is formed to define a number of axially spaced ridges 22 circumscribing its outer surface, the ridges 22 extending approximately along directions transverse to the longitudinal axis of the dilator element 12. The ridges 22 dilate and scrape the sidewalls of the bone tunnel as the dilator element 12 is first advanced to define a dilated sidewall and then is retracted to smoothen the sidewall surface to more closely accommodate a bone plug of a prosthesis.

As described above, the dilator 5 is used, for example, in knee repair surgery such as in ACL replacement. A surgeon drills a small hole through a bone (e.g., the upper portion and through the condyle of the femur) and is followed by a larger coaxial hole that may not extend fully through the bone. A drill bit is removed and leading portion 10L of the of the dilator 5 is then inserted into the bone hole with the leading end 10L extending all the way through the smaller hole and out of the patient's body. The dilator device 12 is advanced until the conical portion 14 reaches the entry of the typically round drill hole. A slap hammer is then attached to the leading segment 10L of the shaft 10 and a cone section is pulled, in increments, into the round hole. By repetitive hammering, the portion of the device 5 that transitions from a cone shape to a rectangular shape also gets pulled into the drill hole, thus changing the shape of the drill hole from round to rectangular or other predefined shape. During the hammering process, with each pull of the device 5, the bone material deforms progressively into the shape of the rectangular portion of the device 5, as the device 5 is progressively forced deeper into the hole. The resulting rectangular bone hole can receive a rectangular bone graft that results in a better fit to facilitate bone growth and incorporation of the graft into the native bone. The device 5 is removed by engaging the trailing portion 10T of the shaft and pulling or hammering in the reverse direction. It should be understood that although the illustrative embodiment is described as reshaping the tunnel to a rectangular or oval cross section, the invention may be used to dilate bone tunnels to other geometric configurations.

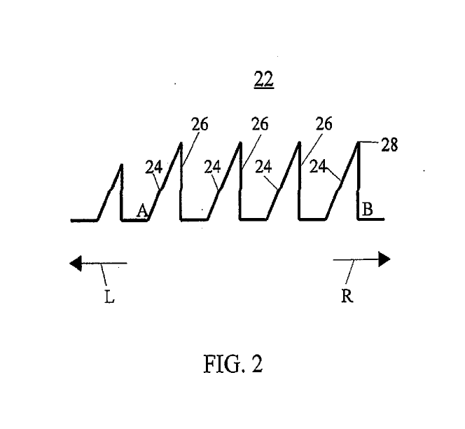

FIG. 2 illustrates the ridges 22 of FIG. 1 in an enlarged, diagrammatic cross section. By way of example, the ridges 22 preferably are formed to have insertion and removal faces 24, 26, respectively.

The insertion faces 24 face generally in a leading direction L and the removal faces 26 face generally in a removal direction R. The insertion and removal faces 24, 26 meet at a ridge 28, which preferably is relatively sharp. The insertion faces 24 preferably make an angle greater A than 90 degrees with a longitudinal axis of the device 5 and the removal faces 26 preferably make an angle B of 90 degrees or less with the axis. A slope of the insertion face 24 facilitates insertion while the angle B of the removal face 26 enables the ridge to act as somewhat of a scraper to smoothen and clean debris from the dilated tunnel wall as the device is removed.

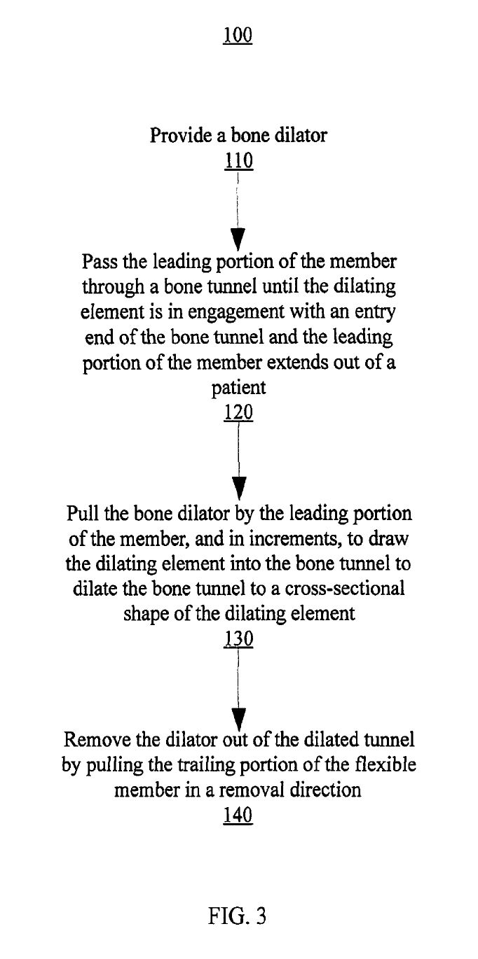

As shown in FIG. 3, a process 100 of dilating a bone tunnel includes providing (110) a bone dilator having an elongate, flexible member having leading and trailing portions and a dilating element disposed on the member, intermediate ends of the flexible member.

Process 100 passes (120) the leading portion of the member through a bone tunnel until the dilating element is in engagement with an entry end of the bone tunnel and the leading portion of the member extends out of a patient.

Process 100 pulls (130) the bone dilator by the leading portion of the member, and in increments, to draw the dilating element into the bone tunnel to dilate the bone tunnel to a cross-sectional shape of the dilating element.

Process 100 removes (140) the dilator out of the dilated tunnel by pulling the trailing portion of the flexible member in a removal direction.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

What is claimed is:

1. A bone tunnel dilator comprising:
an elongate, flexible member having leading and trailing portions; and
at least one dilating element disposed on a member intermediate the leading and trailing ends, a leading end of the shaft designed to be inserted through a bone tunnel and the at least one dilating element pulled into an entry end of the bone tunnel by applying pulling force to the leading end of the shaft to dilate an entry end of the bone tunnel to a shape and size defined by the dilating element, the dilator removed by pulling on the trailing end of the shaft.

2. The bone tunnel dilator of claim 1 wherein the dilating element has a plurality of ridges spaced axially along a portion of the dilating element, the ridges circumscribing the dilating element and defining a predetermined cross-section of the bone tunnel to be dilated.

3. The bone tunnel dilator of claim 2 wherein the ridges have insertion and removal faces, the insertion faces being arranged to facilitate insertion of the dilator into the bone tunnel and the removal faces being arranged to smoothen a surface of a dilated tunnel wall.

4. The bone tunnel dilator of claim 1 wherein the dilating element includes a smooth surface circumscribing the dilating element and defining a predetermined cross section of the bone tunnel to be dilated.

5. The bone tunnel dilator of claim 1 wherein the dilator element includes two dilating elements, the first dilator including a smooth surface and the second dilator including a surface with ridges.

6. The bone tunnel dilator of claim 1 where the dilator element is modular to the shaft portion and interchangeable.

7. The bone tunnel dilator of claim 1 wherein the member is suture.

8. The bone tunnel dilator of claim 1 where the member is composed of two different materials.

9. A method of dilating a bone tunnel comprising:
providing a bone dilator having an elongate, flexible member having leading and trailing portions and a dilating element disposed on the member, intermediate ends of the flexible member;
passing the leading portion of the member through a bone tunnel until the dilating element is in engagement with an entry end of the bone tunnel and the leading portion of the member extends out of a patient;
pulling the bone dilator by the leading portion of the member, and in increments, to draw the dilating element into the bone tunnel to dilate the bone tunnel to a cross-sectional shape of the dilating element; and
removing the dilator out of the dilated tunnel by pulling the trailing portion of the flexible member in a removal direction.

10. The method of claim 9 wherein the dilating element has a plurality of ridges spaced axially along a portion of the dilating element, the ridges circumscribing the dilating element and defining a predetermined cross section of the bone tunnel to be dilated.

11. The method of claim 10 wherein the ridges have insertion and removal faces, the insertion faces being arranged to facilitate insertion of the dilator element into the bone tunnel and the removal faces being arranged to smoothen a surface of a dilated tunnel wall.

12. The method of claim 9 wherein the dilating element includes a smooth surface circumscribing the dilating element and defining a predetermined cross-section of the bone tunnel to be dilated.

13. The method of claim 9 wherein the dilator element includes two dilating elements, the first dilator including a smooth surface and the second dilator including a surface with ridges.

14. The method of claim 9 where the dilator element is modular to the flexible member and interchangeable.

15. The method of claim 9 wherein the flexible member is suture.

16. The method of claim 1 where the member is composed of two different materials.
